(19) United States
(12) Patent Application Publication
(10) Pub. No.: US 2026/0031207 A1
(43) Pub. Date: Jan. 29, 2026

(54) SYSTEM FOR REDUCING MATERNAL MORTALITY AND METHOD OF USE THEREOF

(71) Applicant: Morehouse School of Medicine, Atlanta, GA (US)

(72) Inventors: Natalie HERNANDEZ, Atlanta, GA (US); Andrea G. Parker, Decatur, GA (US); Rasheeta Chandler, Stone Mountain, GA (US)

(21) Appl. No.: 19/347,102

(22) Filed: Oct. 1, 2025

Related U.S. Application Data
(63) Continuation of application No. 17/935,386, filed on Sep. 26, 2022.

(51) Int. Cl. G16H 10/65; G16H 10/20

(52) U.S. Cl. CPC G16H 10/65; G16H 10/20

(57) ABSTRACT

A method for monitoring and/or modifying behavior to reducing maternal mortality risk, including the steps of receiving, via a user interface of an application executing on one or more computer processors, a self-surveillance module, wherein said self-surveillance module comprises inputs from the subject for personal information for a plurality of types of information selected from the types of information comprising doctor appointments, physiologic parameters, mental health survey results, indicators of social instability and personally recorded expression; determining, via the one or more computer processors, a total abnormality point value of trends for in the subject via the one or more computer processors, based on the sum total of abnormality point values in real-time, wherein when the total abnormality point value of the trends for the subject exceeds a pre-selected point value an abnormality alert is generated.

This application is a Continuation of U.S. application Ser. No. 17/935,386, filed Sep. 26, 2022. The entirety of the aforementioned applications is incorporated herein by reference.

### FIELD

The present application relates generally to a system and method for monitoring and improving behavior and reducing maternal mortality risks.

### BACKGROUND

Postpartum maternal health care is an especially neglected component of women's overall health care; the weeks following birth are a critical period (for both mothers and infants) for setting the stage for long-term health and well-being. Despite this, as many as 40% of women do not attend postpartum visits. Not attending a postpartum visit is higher among Black and low-income women, those with public insurance, and those living in rural areas. The consequences of inattention to postpartum care can be seen in the aforementioned maternal morbidity and mortality statistics, which highlight missed opportunities to enhance healthcare among postpartum women.

Mobile health (mHealth) technology has the potential to overcome barriers and gaps in postpartum care. Mobile phone applications (apps) have grown increasingly popular as sources of health information. Given the near-ubiquitous use of mobile phones in the United States, mHealth apps offer scalability and expansion to certain audiences that may otherwise be unreached. Recent studies have demonstrated that postpartum women frequently use mobile phones and internet to search for health information. Though participants reported using apps throughout their pregnancy, they also reported that apps were unavailable or did not address their concerns during the postpartum period. This highlights the need for apps that provide postpartum content to rural ethnic minority women, i.e. African American women, who face unique challenges during the postpartum period.

Currently, there are not sufficient systems in place to effectively deal with this problem. Indeed, policy-makers and private sector organizations typically lack effective tools to objectively assess their own deficiencies and address the problems. Racial maternal morbidity and mortality disparities among African American women are derivatives of myopic prevention research that primarily focuses on individual-level interventions while discounting the systems, processes, and cultural factors that contribute to high rates of maternal morbidity and mortality among African American women. This application provides an effective approach to meeting these needs.

### SUMMARY

An aspect of the application is a method for monitoring and reducing maternal mortality in a subject, comprising the steps of: (a) receiving, via a user interface of an application executing on one or more computer processors, a self-surveillance module, wherein said self-surveillance module comprises inputs from the subject for personal information for a plurality of types of information selected from the types of information comprising doctor appointments, physiologic parameters, mental health survey results, indicators of social instability and personally recorded expression; (b) storing, via the one or more computer processors, said personal information of said subject in a database accessible by said application, and accessible by said subject via said user interface of said application; (c) receiving, via said user interface of said application executing on one or more computer processors, a data visualization dashboard, wherein said dashboard displays to said subject trends over time in said personal information stored in said database; (d) receiving, via a user interface of an application executing on one or more computer processors, a goal-setting module, wherein said goal-setting module comprises inputs from the subject regarding personal performance targets that are desired to be achieved in connection with trends in those types of personal information stored in said database; (e) assigning, via the one or more computer processors, an abnormality point value for any of a plurality of deviations from trends over time in said personal information stored in said database and an abnormality point matrix stored on a memory device accessible by the one or more computer processors, the abnormality point matrix being generated and continually updated by a machine learning module; (f) determining, via the one or more computer processors, a total abnormality point value of said trends for said subject via the one or more computer processors, based on the sum total of abnormality point values in real-time, wherein when the total abnormality point value of said trends for said subject exceeds a pre-selected point value an abnormality alert is generated; (g) notifying, via said user interface, said subject about the abnormality alert; and (h) providing, via said user interface, health information messaging to said subject, wherein said health information messaging is tailored to undo said abnormality in said trends of said subject.

In certain embodiments, the user interface is accessed by said subject on a mobile device.

In certain embodiments, the user interface is accessed by said subject on a wearable device.

In certain embodiments, the health information messaging to said subject comprises one or more type of messaging including self-tracking reminders, doctor appointment reminders, alerts to notify a healthcare provider and de-stress providers.

In certain embodiments, the health information messaging is triggered automatically in response to trends in said personal information of said subject that indicates increased maternal mortality risk.

In certain embodiments, the health information messaging is displayed on said user interface as a text alert to said subject.

In certain embodiments, the health information messaging is displayed on said user interface as a representative symbol to said subject, wherein said symbol alerts said subject to availability of health information that is accessible via said user interface.

In certain embodiments, the method further comprises the step of: (i) receiving, via a user interface of an application executing on one or more computer processors, messages indicating that said subject is within a predefined distance of a healthcare provider and providing information regarding how said subject can travel to said healthcare provider.

In certain embodiments, the messages include geo-location information for said healthcare provider.

In certain embodiments, the self-surveillance module further comprises inputs from the subject for personal information for social support selected from the types of information comprising sleep deprivation, absence of assistance with baby or other children, and mood-related symptoms, wherein said subject inputs measurements of personal stress on a predefined measurement scale presented to said subject via said user interface.

In certain embodiments, the health information messaging comprises stress management strategies that respond to the trends in personal information for social support of said subject.

In certain embodiments, the method further comprises the step of: (j) receiving, via a user interface of an application executing on one or more computer processors, messages inviting said subject to join groups of other individuals using said application, wherein said groups are able to communicate amongst members of the group via the user interface.

In certain embodiments, the method further comprises the step of tracking over time, via the one or more computer processors and the total abnormality point values, the relative improvement or lack of improvement over time with respect to maternal mortality risk by said subject.

In certain embodiments, the method further comprising the step of providing a system comprising: (i) one or more computing devices in data communication with each other, each device having one or more computer processors, a data communication connection, and one or more tangible non-transitory computer-readable media accessible by the one or more computer processors, and (ii) a plurality of databases, including a personal information database and a health information messaging database, wherein a personal information database and a health information messaging database are each stored in the one or more tangible non-transitory computer-readable media.

In certain embodiments, the plurality of databases further comprises a resources database.

In certain embodiments, the resources database comprises multi-media information, wherein said multi-media information is accessible by said subject via said user interface.

Another aspect of the application is a system for monitoring and reducing maternal mortality in a subject, comprising: one or more computer processors; and one or more tangible computer readable media accessible by the one or more computer processors, wherein the one or more tangible computer readable media comprise instructions that, when executed by the one or more processors, cause the one or more processors to perform: (a) receiving, via a user interface of an application executing on one or more computer processors, a self-surveillance module, wherein said self-surveillance module comprises inputs from the subject for personal information for a plurality of types of information selected from the types of information comprising doctor appointments, physiologic parameters, mental health survey results, indicators of social instability and personally recorded expression; (b) storing, via the one or more computer processors, said personal information of said subject in a database accessible by said application, and accessible by said subject via said user interface of said application; (c) receiving, via said user interface of said application executing on one or more computer processors, a data visualization dashboard, wherein said dashboard displays to said subject trends over time in said personal information stored in said database; (d) receiving, via a user interface of an application executing on one or more computer processors, a goal-setting module, wherein said goal-setting module comprises inputs from the subject regarding personal performance targets that are desired to be achieved in connection with trends in those types of personal information stored in said database; (e) assigning, via the one or more computer processors, an abnormality point value for any of a plurality of deviations from trends over time in said personal information stored in said database and an abnormality point matrix stored on a memory device accessible by the one or more computer processors, the abnormality point matrix being generated and continually updated by a machine learning module; (f) determining, via the one or more computer processors, a total abnormality point value of said trends for said subject via the one or more computer processors, based on the sum total of abnormality point values in real-time, wherein when the total abnormality point value of said trends for said subject exceeds a pre-selected point value an abnormality alert is generated; (g) notifying, via said user interface, said subject about the abnormality alert; and (h) providing, via said user interface, health information messaging to said subject, wherein said health information messaging is tailored to undo said abnormality in said trends of said subject.

In certain embodiments, the user interface is accessed by said subject on a mobile device.

In certain embodiments, the system further comprising the step of tracking over time, via the one or more computer processors and the total abnormality point values, the relative improvement or lack of improvement over time with respect to maternal mortality risk by said subject.

Another aspect of the application is a tangible non-transitory computer readable storage medium, comprising instructions that, when executed by a computer processor, cause the processor to: (a) receiving, via a user interface of an application executing on one or more computer processors, a self-surveillance module, wherein said self-surveillance module comprises inputs from the subject for personal information for a plurality of types of information selected from the types of information comprising doctor appointments, physiologic parameters, mental health survey results, indicators of social instability and personally recorded expression; (b) storing, via the one or more computer processors, said personal information of said subject in a database accessible by said application, and accessible by said subject via said user interface of said application; (c) receiving, via said user interface of said application executing on one or more computer processors, a data visualization dashboard, wherein said dashboard displays to said subject trends over time in said personal information stored in said database; (d) receiving, via a user interface of an application executing on one or more computer processors, a goal-setting module, wherein said goal-setting module comprises inputs from the subject regarding personal performance targets that are desired to be achieved in connection with trends in those types of personal information stored in said database; (e) assigning, via the one or more computer processors, an abnormality point value for any of a plurality of deviations from trends over time in said personal information stored in said database and an abnormality point matrix stored on a memory device accessible by the one or more computer processors, the abnormality point matrix being generated and continually updated by a machine learning module; (f) determining, via the one or more computer processors, a total abnormality point value of said trends for said subject via the one or more computer processors, based on the sum total of abnormality point values in real-time, wherein when the total abnormality point value of said trends for said subject exceeds a pre-selected point value an abnormality alert is generated; (g) notifying, via said user interface, said subject about the abnormality alert; and (h) providing, via said user interface, health information messaging to said subject, wherein said health information messaging is tailored to undo said abnormality in said trends of said subject.

### BRIEF DESCRIPTION OF THE DRAWINGS

The present invention can be better understood by reference to the following drawings, wherein like references numerals represent like elements. The drawings are merely exemplary to illustrate certain features that may be used singularly or in any combination with other features and the present invention should not be limited to the embodiments shown.

FIG. 1 shows an exemplary flow chart overview of the personalization survey and its relationship to data analytics.

FIG. 2 shows an exemplary flow chart overview of how data analytics relates to the generation of personal health care insights.

FIG. 3 shows an exemplary flow chart showing how data analytics relates to the use of non-clinical health factors, such a mood, identity and relationships, to generate personal health care insights.

FIG. 4 shows an exemplary flow chart showing how data analytics relates to personalized data presentation based on personalized insights and consequent personal goal-setting.

FIG. 5 shows an exemplary flow chart showing how data analytics relates to personalized gamification to incentivize positive and pro-social behaviors.

FIG. 6 shows an exemplary embodiment of a sign-in screen.

FIG. 7 shows an exemplary embodiment of a log-in screen.

FIG. 8 shows an exemplary embodiment of a resources screen.

FIG. 9 shows an exemplary embodiment of a health screen.

FIG. 10 shows an exemplary embodiment of a mental health screen.

FIG. 11 shows an exemplary embodiment of an emoji tracking screen.

FIG. 12 shows an exemplary embodiment of a mental health assessment screen.

FIG. 13 shows an exemplary embodiment of a physical activity screen.

FIG. 14 shows an exemplary embodiment of a sleep screen.

FIG. 15 shows an exemplary embodiment of a weight screen

FIG. 16 shows an exemplary embodiment of a blood pressure screen

FIG. 17 shows an exemplary embodiment of a health information screen

FIG. 18 shows an exemplary embodiment of a calendar screen

### DETAILED DESCRIPTION

The following detailed description is presented to enable any person skilled in the art to make and use the object of this application. For purposes of explanation, specific nomenclature is set forth to provide a thorough understanding of the present application. However, it will be apparent to one skilled in the art that these specific details are not required to practice the subject of this application. Descriptions of specific applications are provided only as representative examples. The present application is not intended to be limited to the embodiments shown, but is to be accorded the widest possible scope consistent with the principles and features disclosed herein.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising”, “includes”, “including”, “has”, “have”, “having”, “with” and the like, when used in this specification, specify the presence of stated features, integers, steps, operations, elements, and/or components, but do not preclude the presence or addition of one or more other features, integers, steps, operations, elements, components, and/or groups thereof.

As used in this application, the terms “component”, “module”, “system”, “interface”, and the like are generally intended to refer to a computer-related entity, either hardware, a combination of hardware and software, software, or software in execution. For example, a component may be, but is not limited to being, a process running on a processor, a processor, an object, an executable, a thread of execution, a program, or a computer. By way of illustration, both an application running on a controller and the controller can be a component. One or more components residing within a process or thread of execution and a component may be localized on one computer or distributed between two or more computers. As used herein, the term “wireless” means any wireless signal, data, communication, or other interface including without limitation Wi-Fi, Bluetooth, 3G, 4G, HSDPA/HSUPA, TDMA, CDMA (e.g., IS-95A, WCDMA, etc.), FHSS, DSSS, GSM, PAN/802.15, Wi-MAX (802.16), 802.20, narrowband/FDMA, OFDM, PCS/DCS, analog cellular, CDPD, satellite systems, millimeter wave or microwave systems, acoustic, and infrared (i.e., IrDA).

As used herein, the terms “Internet” and “internet” are used interchangeably to refer to inter-networks including, without limitation, the Internet.

As used herein, the term “memory” includes any type of integrated circuit or other storage device adapted for storing digital data including, without limitation, ROM. PROM, EEPROM, DRAM, SDRAM, DDR/2 SDRAM, EDO/FPMS, RLDRAM, SRAM, “flash” memory (e.g., NAND/NOR), and PSRAM.

As used herein, the term “computer processor” refers generally to all types of digital processing devices including, without limitation, digital signal processors (DSPs), reduced instruction set computers (RISC), general-purpose (CISC) processors, microprocessors, gate arrays (e.g., FPGAs), PLDs, reconfigurable compute fabrics (RCFs), array processors, and application-specific integrated circuits (ASICs). Such digital processors may be contained on a single unitary IC die or distributed across multiple components.

Prevent Maternal Mortality using Mobile technology (PM3) is a maternal mortality prevention and optimal reproductive health promotion mobile app created to ultimately increase postpartum comorbidity self-management, and to ensure access to social support and community resources. An aspect of the present application is to standardize postpartum discharge education and create a sustainable mechanism to disseminate postpartum resources to women who are most impacted by maternal morbidity and mortality in remote locations. The present application describes a culturally relevant mobile app that reduces barriers for postpartum women and allows them to access all relevant postpartum information and resources at home. The methods and systems herein are focusing exclusively on the postpartum transition rather than the myriad of current apps that focus on pregnancy. The methods and systems herein are primed to modernize postpartum discharge education and postpartum transition self-management.

Previous studies investigating cortisol levels in marginalized communities have relied heavily upon blood, urine, or salivary cortisol samples. These measures are subject to influence by daily variations in hypothalamic pituitary adrenal axis activity and environment circumstances as they assess only momentary, or at most, 24 hours of cortisol exposure. Therefore, findings from these studies may not accurately reflect relationships between mental health, stressful events, and long-term cortisol levels. Cumulative cortisol over several months is a better measure of overall cortisol levels and can be easily obtained using hair samples. Investigating the utility of hair cortisol concentrations as a chronic stress biomarker may inform interventions and improve understanding of the long-term impact of multifaceted stressors experienced by rural Black women. The methods and systems herein provide a significant improvement over previous healthcare approaches for marginalized communities.

PM3 includes an integrated self-management and reproductive health messaging feature having: a. detecting both abnormality and triggering a response if an abnormality is detected; b. recording i. doctor appointments, ii. PA, iii. physiologic parameters [i.e. blood pressure], iv. mental health survey results (i.e. race related stress), v. indicators of social instability (e.g., changes in employment), vi. descriptive anecdotes in the journal section of PM3 Self-management; c. to encourage use of these self-management features, a key part of the formative evaluation process is to investigate women's values around self-tracking data and the insights they would most like to gain from this data; d. a goal-setting feature will enable women to choose from maternal health goals (e.g., stress reduction) and manage progress toward those goals; e. the self-management data (e.g., symptoms, behavioral, attitudinal, and social instability indicator data) is used to drive tailored, adaptive messaging that prevents maternal morbidity (e.g., self-tracking reminders, doctor appointment reminders, alerts to notify a healthcare provider, de-stress techniques). PM3 will allow women to schedule tasks and reminders to facilitate adherence to risk reduction behaviors (e.g., medication and medical visit adherence).

PM3 includes a support feature having: a. GPS locator for local providers who can provide postpartum healthcare education and/or services (e.g. doulas, doctors, etc.). PM3 can provide a centralized place for identifying healthcare providers and facilities. Geofencing capability, a location-based service, sends messages to participants when they enter predefined locations (e.g. 1-mile radius) that have facilities for women's health, family planning, and/or mental health care; b. At-home support tracking. Women will track social support gaps that provoke stress (e.g. sleep deprivation, no assistance with baby or other children, as well as mood-related symptoms (e.g. anxiety). Women will be able to indicate “no social support,” “minimal social support,” or “overwhelming social support” and rate their stress from 0 (no stress) to 5 (extremely stressed); c. The app will respond with stress management strategies: i. A virtual social support group for the mothers, stratified by county, will occur in a monitored chat room. The app provides a virtual environment for members to connect, extending opportunities for peer modeling, learning, and support online; ii. shares maternal health goals they are pursuing in the app, use messaging features to provide support around these goals and in their lives more broadly, and share insights through the educational messaging components to support peer learning; and d. user's selected PM3-affiliated members who will have limited access to user-initiated tasks, reminders, and appointments. PM3 features also supplement the self-Management features by allowing users to directly engage with health professionals and trusted maternal health organizations through direct messaging. Also, the support features include moderated group discussion that will be led by health professionals, trusted maternal health organizations, and vetted community members.

PM3 resources will make available to participants: a multimedia resource kit comprising videos, current events, news feeds, podcasts, and support groups related to women's postpartum health and community resources (e. g. housing, health services, etc.). The application will provide materials like 1-2 minute video clips and infographics. To encourage use of these resources, the application will recommend specific content through a “Resource Spotlight” feature that suggests content based on data collected through the PM3 self-management feature. By associating semantic metadata with each resource in the application's database, the technology will be able to suggest relevant resources based on trends in each woman's data. Suggested resources will adapt over time as users' logging (e.g., of behaviors, attitudes, and social stressors) evolves. Using a crowdsourcing approach, participants will be able to report on the GPS feature any community resources that might be relevant to their peers, such as community health fairs, food banks, and affordable childcare options.

The users will have the ability to interact with resource content through a range of pre-populated, positive through negative, emojis and by sending feedback to the research team. These emoji interaction and feedback from the content shared will help to define what is deemed valuable by the users.

The application will update women on maternal and reproductive health policies that impact women. Participants will be provided with strategies to confidently engage with the healthcare system (e.g., questions to ask providers) and address community-level factors that perpetuate social inequity (e.g. digital employment training opportunities, interview skill building, etc.).

PM3 also has features that address the psychological needs for competence (e.g., goal progress visualization in self-management), autonomy (e.g., ability to customize one's app avatar), and relatedness (e.g., community building features). PM3 provides tailored interventions based on a profile of adherence to postpartum medical visits, and integration monitoring of sleep, heart rate variability, stress, weight, and goal setting. Wearable technology may be integrated with the application to enhance self-monitoring.

One of ordinary skill will understand that although the particular focus of the methods and systems described herein are on improving care for a target population of rural Black women, the method and systems described herein are not limited in scope to a particular population. For example, the methods and systems described herein may be used, without limitation, in the context of rural Indigenous populations, generationally impoverished rural White populations, or under-served urban populations, amongst other groups.

### Personalization

The application uses a broad spectrum of patient generated data that assists with self-management; in a specific embodiments, the application is provided to women at discharge from their hospital early in the post-partum period. The application addresses the failure to provide women who are discharged with information that is valuable to them in a manner that is culturally, locally or personally relevant. The application presents data and perform data analytics so as to support experiential learning. The users enter a broad spectrum of data that addresses both clinical and non-clinical needs, and data analytics, e.g., machine learning, is used to surface meaningful personalized insights. The application can be used as a means of communication to health care providers, and can be accessed by providers to monitor data trends in patients, including sending health alerts if need be.

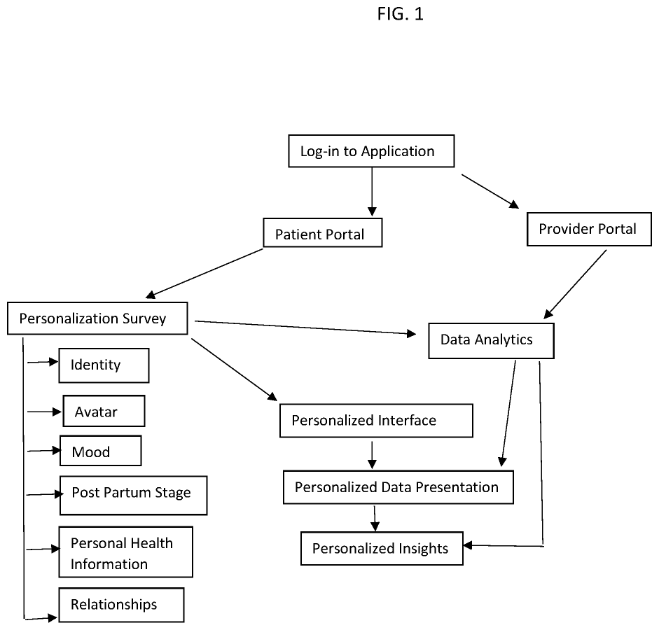

An aspect of the application is a strong personalization component. Initially, a user will enter the application and then fill out a personalization survey (see FIG. 1) asking personal information about health history, postpartum stage, identity, mood, relationships (family and/or other), as well as asking the user to select and/or build a personal avatar that will function as a representation of the user themselves within the application. The application can present and receive data in different forms, e.g., use of emojis to represent patient mood whether by input selection by the patient or presentation of trends within the application.

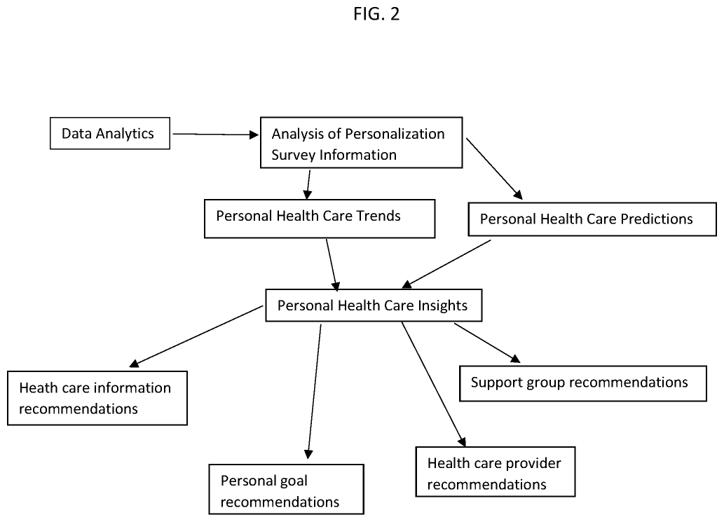

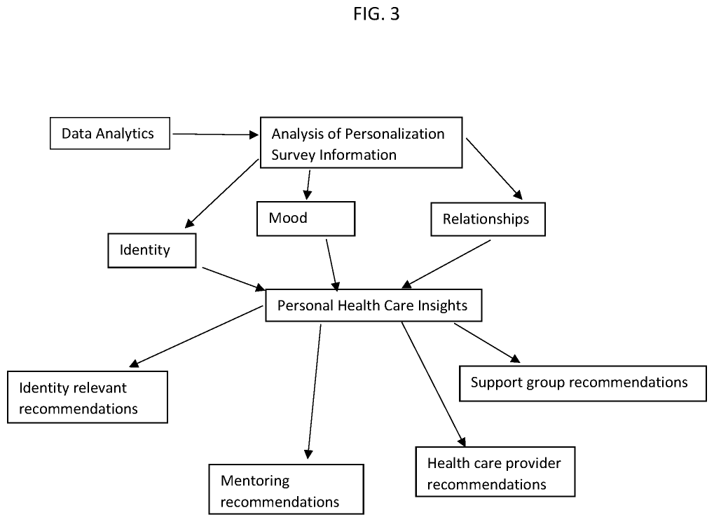

Based on responses to the personalization survey, such as information about existing health conditions, the particular stage of postpartum period the user is at, etc., the application uses data analytics (see FIG. 2) to provide personalized data presentation of the resources in the application to the user via the personalized interface. In particular, by analyzing not only the clinical health data provided by the user (e.g., blood pressure, any preexisting conditions, asthma, etc.), but also analyzing the patient's emotional state (e.g., mood) and relationship and identity statuses (see FIG. 3), the application is able to provide personalized insights to the user based on a personalized set of resources recommended to them from within the application. This serves to support them in postpartum period and onwards.

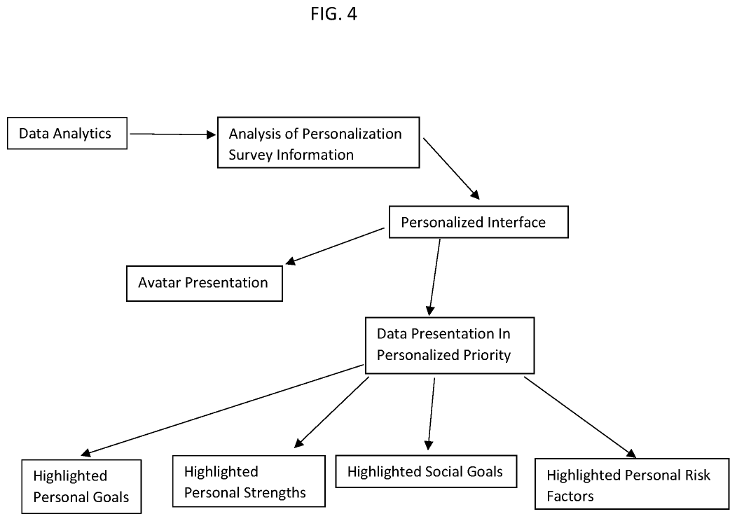

The personalization performed by the application is more sophisticated than currently provided to patient populations, which often try to solely respond to clinical health inputs (e.g., blood pressure, weight, and so on), by integrating mental health, identity and relationship into the analysis of the patient, the application provides more effective culturally relevant and locally relevant information to the user. In certain embodiments, analysis of data streams through use of machine learning predictions to be made concerning health conditions, including potential positive or negative outcomes, or possible plateauing of health improvements (see FIG. 4). In particular embodiments, mood inputs or relationship status may be cross-checked by the application with blood pressure, or other health conditions, and personalized insights may be provided that are designed to address possible issues, such as, e.g., depression. The application is also capable of predicting when certain combinations of mood and positive trends in clinical health factors are likely to produce positive outcomes, and produce personalized insights that affirm and encourage the user to maintain effective behavioral trends. In particular, the application provides access to social support groups formed from other users. A user may be assigned a particular social support group based on shared identity factors (e.g., religion, sexual orientation, age group, etc.) or common health factors (e.g., trying to quit smoking).

The application may assign particular individuals as mentors based on tracking their interaction with other users, e.g., an individual who actively participates in support groups and who other users positively respond to as evidenced by improvements in their health and mood trends may be selected to play a mentoring role. For example, individuals who are repeatedly not reaching personalized health goals may be assigned a mentor who has experienced similar negative trends in the past, but has overcome them. The application also uses data analytics of using users interactions within the system to provide a personalized interface that raises underused resources to the user, and can also prompt private encouragement from another user.

Although the application is patient-centered, physicians are enabled to interact with patients through a provider portal. The application can provide data analytics that can be useful for providers, such as, e.g., support persons, patient navigators, doulas, etc. The data analytics provided to health care providers may be integrated with electronic health records. The application may also be adapted to different languages and foreign countries. For example, the application may be integrated with a doula practice to provide technically augmented doula care. A doula will have a support partner interface, which will enable direct real-time communication with the user, provide alerts regarding mood and health trends (including positive trends and prompts for positive affirmation of the user), as well as a dashboard of clinical health data. Instead, of generic discharge instructions, providers can use the application as a discharge system to engage patients beyond going home. A provider would have discretion, if a person is high risk, to provide additional monitoring using a wearable device with remote health monitoring option.

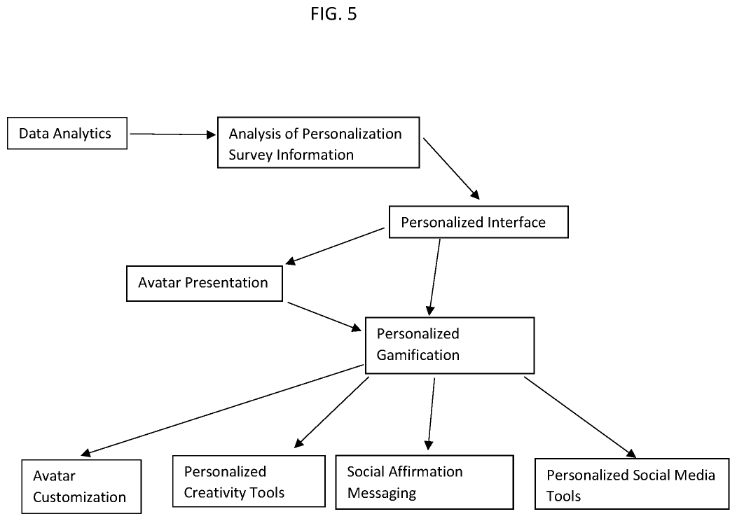

An aspect of the application is provision of behavioral incentives through gamification (see FIG. 5). For example, gamification can occur through multiple levels, such as badges in personal profile viewable by others, recordation and display of personal streaks and personal achievements (such as reaching a personalized health goal, monitoring goals, behavioral changes, etc.). In certain circumstances, users are enabled to respond to or to engage in certain activities through use of social rewards, e.g., storybook chapters are unlocked, different support room are unlocked, social features are unlocked that can celebrate a positive event (e.g., throwing confetti, sharing music, positive memes, etc.). Data analytics can also be used to highlight such features to users who are likely to interact in this manner.

### Sign-Up

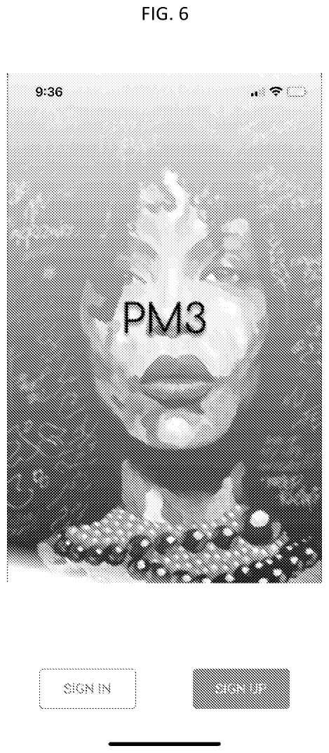

FIG. 6 shows an exemplary embodiment of a sign-in screen. In certain embodiments, the sign-up module comprises one or more of the following features selected from amongst email address, password, participant ID, terms and conditions, group participation guidelines, progress bar, profile, demographic questionnaire, health survey, and completion notification. In certain embodiments, the user will enter an email address. In certain embodiments, the user will create a unique username. In certain embodiments, the user will create a strong password twice (a strong password is a password comprising a plurality from mixed numbers and characters, upper and lower case letters and special characters so as to be hard to detect by either humans or computers); the second password entry is for verification. In certain embodiments, a strong password is necessary for further access to modules of the mobile application.

In certain embodiments, users will be given a unique participant code to use for account creation. In certain embodiments, users will not be allowed to continue in the sign-up process without a study ID. In certain embodiments, the sign-up module shall support the generation of a unique participant ID for each participant, enable support staff to access that ID, and that ID with account sign up information will be sent to the participant. In certain embodiments, the app shall require a participant ID at sign-up

In certain embodiments, users will be able read full Terms and Conditions form and must agree to Terms and Conditions in order to use the app. In certain embodiments, check box on sign-up screen to indicate agreement with Terms and Conditions; user must check the box. In certain embodiments, the module shall require participants to agree to the terms and conditions by selecting a checkbox at the sign-up page. In certain embodiments, the module shall allow users to access the Terms and Conditions after sign-up.

In certain embodiments, users need to be able to easily see the expectations for posting messages in the Settings section. In certain embodiments, these guidelines should be viewable in the Groups section and should be included as a section within the Terms and Conditions. In certain embodiments, the module shall enable access to the specific group participation guidelines in a page that is separate from the Terms and Conditions.

In certain embodiments, user will be able to view their progress through the Sign-Up process in a “Progress Bar.” In certain embodiments, the module will show a progress bar that visualizes user progress through app sign-up process

In certain embodiments, user will be able to create a profile, and specify which aspects of their profile (if any) will be visible to other app users

In certain embodiments, user required to complete all prompts for Demographic Survey as a part of the sign-up process. In certain embodiments, the module provides a scrolling/circular menu to select county of residence, e.g., all counties in a state listed. In certain embodiments, the module provides a scrolling/circular menu to select month of birth; do not require day of birth. In certain embodiments, the module provides a scrolling/circular menu to select year of birth. In certain embodiments, free response field to enter number of children. In certain embodiments, the module provides a scrolling/circular menu to select Month and year of youngest child; do not require day of birth. In certain embodiments, the module will show the demographic survey to users as part of the sign-up process.

In certain embodiments, users will be required to complete all prompts for severe maternal mortality and maternal mortality Risk Assessment survey. In certain embodiments, Prompt 1 will be check all that apply; “none of the above” if an acceptable response. In certain embodiments, Prompt 2 “yes/no.” In certain embodiments, Prompt 3 will be check all that apply. In certain embodiments, the module will show the risk assessment survey to users as part of the sign-up process.

In certain embodiments, after completing the sign-up process the user will receive a thank you message with a check mark. Also, the message will notify the user that their data will be used to customize their app experience. The message is shown in a pop-up window. In certain embodiments, user will tap check mark to progress into app. In certain embodiments, the module will show a message that thanks the user and notifies them that their data will be used to customize the app experience.

### Log-in

In certain embodiments, the log-in module presents the user with features comprising Email address, Password, Two Factor Authentication and Security. In certain embodiments, user enters email address used during registration to login to app. In certain embodiments, user enters email address used during registration to login to app. In certain embodiments, login will persist for a duration consistent with security best practices. In certain embodiments, log-in module shall require an established username to login. In certain embodiments, log-in module shall require an established password to login. In certain embodiments, user shall have the option to click “stay logged in.” In certain embodiments, the log-in module shall require Two Factor authentication. In certain embodiments, the log-in module shall include current best practices for security and privacy during transmission and at rest.

One of ordinary skill will understand that the order of presentation of different screens or areas of subject matter within the application herein does not imply that the same order would be shown to the user. As discussed herein, a user may be shown different aspects of the application based on personalized insights regarding their health care priorities. The various embodiments discussed herein may also be shown together in information presentation to the user of the application which combines and/or highlights specific information. All the different embodiments described herein may be presented to the user (whether a postpartum patient or healthcare provider) with cross-referencing to each other or in parallel, sequentially or simultaneously.

### Main Dashboard

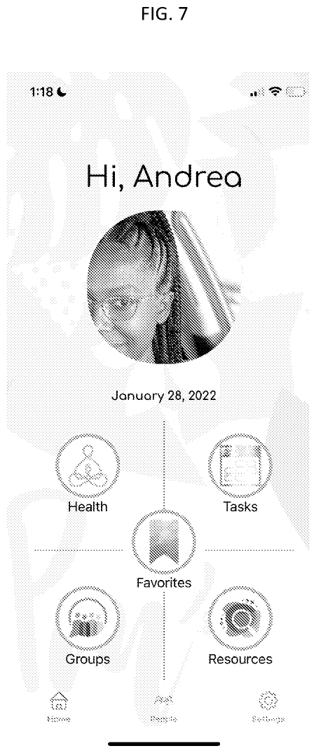

FIG. 7 shows an exemplary embodiment of a main dashboard screen. In certain embodiments, the main dashboard module comprises five icons/features (i.e., Health, Resources, Groups, Task, Collections) are shown around profile picture. In certain embodiments, users are able to access their profile from this screen. In certain embodiments, users need to be able to get a quick overview of their progress towards health goals. In certain embodiments, users need to be able to see when there is new content in the app, so that they are motivated to engage with the various sections of the app. In certain embodiments, new content is defined ideally as content added since the last time the user had a session in the app.

In certain embodiments, the dashboard will include icons for accessing each of the four primary features in the app. In certain embodiments, user will be able to select their profile picture to return to profile screen. In certain embodiments, user will provide circular-visual representation around profile picture that demonstrates progress towards health goals. In certain embodiments, user will be able to see notifications that new content is available in the Tasks, Health, Resources, and Groups sections of the app. In certain embodiments, users can select notifications and be taken to the associated app content.

### Resources

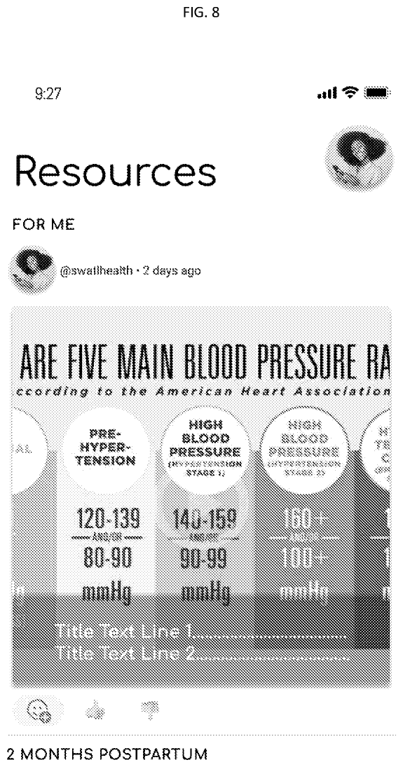

FIG. 8 shows an exemplary embodiment of a resources screen. In certain embodiments, the resources module comprises one or more of the following features selected from amongst Personalized Content Based on Account Setup Questions, Personalized Content Based on Health Self-Monitoring, Discover More/Categorical Resource Lists, Favorites, Content creator username displayed, Reactions/Interactions, View content in app, Shareable Content, Resources Web App, and Support Services Recommendations.

In certain embodiments, users will be able to view customized content, based on their risk assessment and demographic survey. If no risk is indicated, user will receive general app content. In certain embodiments, a section will display as a horizontal scrolling container with content based on survey/self-tracking data. In certain embodiments, a section will display as a horizontal scrolling container with content based on critical points in postpartum period. In certain embodiments, the module will display personalized resources content in two sections, based on the Demographic and Health Survey from sign-up.

In certain embodiments algorithms will decide when tracking in each category (Mental Health, Physical Activity, Sleep, Weight, Blood Pressure) should trigger resources to be highlighted for the user. In certain embodiments, users will be able to view customized content, based on their data entries in the Health section of the app.

In certain embodiments, all app resources are viewable in this section, and are grouped into the same topical categories as they are tagged/stored in the database. In certain embodiments, users can view each category, select the category, and see the available resources in that category in a subsequent screen. In certain embodiments, users will be able to view content grouped by topical category. In certain embodiments, the module displays resources by categorical grouping, e.g., 2×6 grid.

In certain embodiments, bookmark icon is available on each Group and Resource item. Clicking the icon will add the resource to a favorites list. In certain embodiments, users need to be able to easily go back to content of interest to them. Given the number of resources in this system, this is critical. In certain embodiments, the module shall include a “favorites” feature for resource content that allows them to save content to a list for future viewing.

In certain embodiments, content creators should be credited with the videos/resources they developed. In certain embodiments, the name of content creator shall be displayed with content.

In certain embodiments, users need to be able to react/interact with content. In certain embodiments, users need to be able to react/interact with content, voting up/down shows what content the community prioritizes. In certain embodiments, users need to be able to report content. In certain embodiments, users will be able to react to resources using a range of emoji reactions. In certain embodiments, user will have the ability to vote content up or down. In certain embodiments, users will have the ability to report content to the support staff. In preferred embodiments, resource content shall be viewable within the app.

In certain embodiments, users need to be able to share Resources via social media via text, email. In certain embodiments, resource content shall include a “share” button. In certain embodiments, there shall be a web app that enables the team to easily add new resource content to the app. In certain embodiments, the app will provide a list of providers and support services. This list will be easily updatable in the database by support staff admins. In certain embodiments, the app shall include a list of vetted providers and services.

### Groups

In certain embodiments, the groups module comprises one or more of the following features selected from amongst Group List, Group Moderators, Message Violation Tracking, Probation Mode, Request New Group, Join group, Post Messages, View Messages, View Messages as a Feed, Group Notifications, Reactions/Interactions, Messages, and Reply.

In certain embodiments, users will have a grid view of all groups, and a “plus” icon to request a new group. In certain embodiments, users can request that a group contain no more than 10 users. This max threshold should be stored in a database and easily updatable by the support staff admins. In certain embodiments, the module shall include a page of communities/groups. In certain embodiments, groups shall be limited to 10 members. In certain embodiments, users must be able to recognize moderators of the groups. There must be a moderator role that support staff can be granted (this may be synonymous with the admin role). Additionally, there must be a way to assign moderators to each group. In certain embodiments, communities shall clearly show who the moderators are for each group. In certain embodiments, groups need to be monitored for violations of the terms and conditions (e.g., misinformation spreading, harassment, etc.). In certain embodiments, there needs to be a tracking mechanism to determine if users have received three strikes (three violations of terms and conditions with their messages). Users who have reached three strikes will go into probation mode, and will need to have each of their posts reviewed before it is shown. In certain embodiments, the app back-end has an “approved” flag. By default, for all users messages are shown as “true” for approved. If a message is found to be in violation of terms and conditions, then the app can switch flag to “false”. In certain embodiments, the app will track in the back-end the number of terms and conditions violations per user. In certain embodiments, users who are in probation, their messages will be stored by default as “false” for approved, for a period of 30 days. They must be manually changed to “true” by support staff after a review of the content and an assessment that the message adheres to the terms and conditions. In certain embodiments, when a user reaches three strikes (violations), their message will go into moderation-first mode, meaning that they must be reviewed and approved by support staff before made visible to users. In certain embodiments, probation period length should be a variable stored in the database and modifiable by the research team if it determines that the period should be shortened or extended). The value will start out as thirty days. In certain terms, users will be able to request a new group be selecting the “plus” icon on the “Groups” grid. In certain terms, community members should be able to complete an in app form to propose a new group. The proposal should be sent/emailed to the Community moderators to approve or deny the new group proposal.

In certain embodiments, there will be a “join” group button that allows the user to officially become a part of that group. In certain embodiments, user will be able to join a group from a scrollable list of available groups. In certain embodiments, users will be able to post messages in groups. In certain embodiments, users will be able to see messages in groups when entering in each group. In certain embodiments, users will be able to view all new messages in a feed that shows new posts within groups they've joined.

In certain embodiments, users need to be able to know when new posts/comments/reactions/interactions have been added to groups they've joined. This will help drive user engagement with groups. In certain embodiments, group members shall receive in-app notifications that there is new content in groups. In certain embodiments, users will be able to receive group notifications (i.e., new post, new comment on post, reaction/interaction with comment) outside of the app. In certain embodiments, group members shall receive push notifications on their phone (outside of the app) that there is new content in groups.

In certain embodiments, users need to be able to react/interact with content. In certain embodiments, users need a way to see which content is valued most and least highly by the community. In certain embodiments, users need to be able to send feedback about the group messages to the support staff team. In certain embodiments, users need a way to notify the app about inappropriate content they see in the app. In certain embodiments, users will have a range of emoji reactions like Facebook (7 FB emoji selection range). In certain embodiments, user will have the ability to vote content up or down. In certain embodiments, the app will allow users to send feedback about individual messages to the support staff team. In certain embodiments, users will have the ability to report content to the support staff team. In certain embodiments, users will be able to click on links embedded in messages. In certain embodiments, user will have the ability to reply to messages posted.

### Health

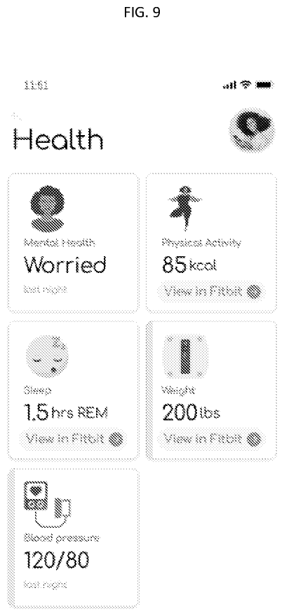

FIG. 9 shows an exemplary embodiment of a health top-level dashboard screen. In certain embodiments, users need to be able to see each area of health self-tracking that is available to them. In certain embodiments, the Health dashboard will have buttons that take users to each of the health tracking areas & display an overview of their data in each health area within this dashboard.

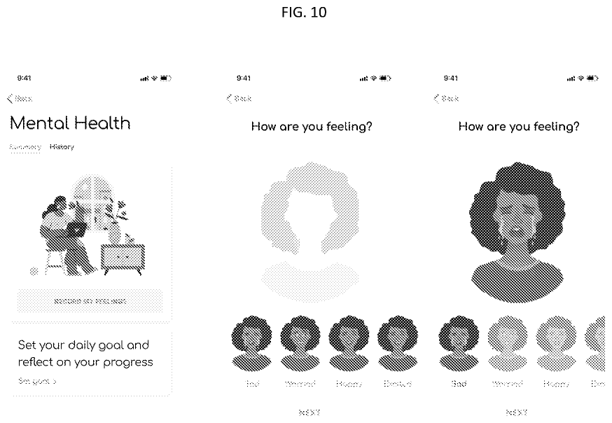

FIG. 10 shows an exemplary embodiment of a mental health dashboard screen. In certain embodiments, the groups module comprises one or more of the following features selected from amongst Mental Health Dashboard-Summary View, Mental Health Dashboard-History View, Mental Health Goal-Setting, Emoji Mood Tracking, Mental Health Assessment. Support staff can determine how the algorithm decides what resources should be shown, and the criteria for determining which resources to show.

In certain embodiments, The Summary view will include the ability to initiate emoji mood tracking. The Summary view will include the ability to see today's tracked mood. The Summary view will include the ability to see highlighted resources based on mood tracking history.

In certain embodiments, users will be able to understand how their moods are changing or staying constant over time. Users will be able to view historical emoji selections. Users will be able to read/watch/listen to past journal entries.

In certain embodiments, user will have the default setting of tracking their mood for 1 week, but will be able to select any goal period (1,2,3 weeks or 1 month). The app will enable users to set a goal to track their mood for a period of time (1 week, 2 weeks, 3 weeks, or 1 month). The app will be recording mood tracking in the database and determining when the user has met their goal. Users will receive a badge when their goal is met.

In certain embodiments, users will be able to select specific support team members that they want to share their goal completion with. When meeting a goal, users will have the option to share with their support team that they've met their goal. User will confirm share of information with support team member before sending. When setting a goal, users will be able to share that goal (e.g., I want to track my mental health for x weeks) with members of the support team.

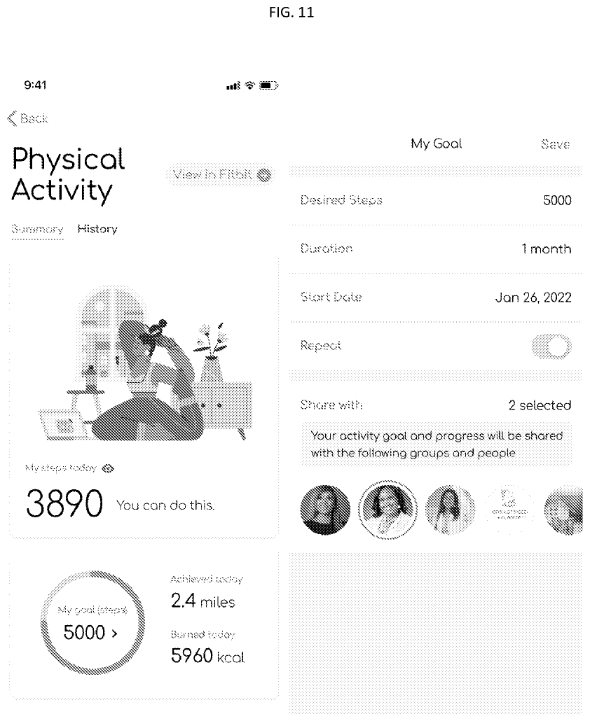

FIG. 11 shows an exemplary embodiment of an emoji mood tracking screen. In certain embodiments, users will be able to complete emoji mental health assessment. This will occur by the user selecting the emoji which represents their feelings/emotion. Users will be able to log their mood by selecting from a set of emojis. Users have the ability to engage in unlimited changing/editing of their emoji selection. Emojis that are selected and unchanged for 5 minutes will be logged as an emoji entry. After logging emoji the user will be able to “journal” emotion. Users will be able to “journal” their emotions using free text, video, or audio journaling.

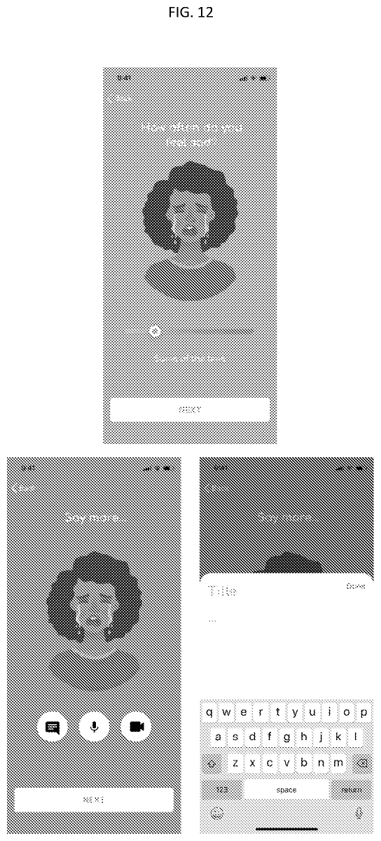

FIG. 12 shows an exemplary embodiment of mental health tracking screen. The support staff will provide parameters for the algorithm for determining if an alert message is needed, and the message to display. The app will display an alert message if users log data of clinical concern.

In certain embodiments, completing the mental health screening will be optional. Users will be able to complete the initial mental health assessment; contingent upon positive/affirmative responses to initial mental health assessment the user will be prompted to complete the further mental health assessment. When affirmative responses are given for the initial mental health assessment, the user will be prompted to complete the further mental health assessment. The support staff may set parameters for the algorithm for determining alerts. Users will complete initial and further mental health assessment at the first time logging emoji mental/emotional health. Next initial and further mental health assessment will be 5 weeks later, then 3-, 9-, and 12-months. Initial mental health assessment will be shown to users at defined time points only. The support staff will set parameters for the algorithm for determining if an alert message is needed, and the message to display. The app will display an alert message if users log mental health assessment data of clinical concern.

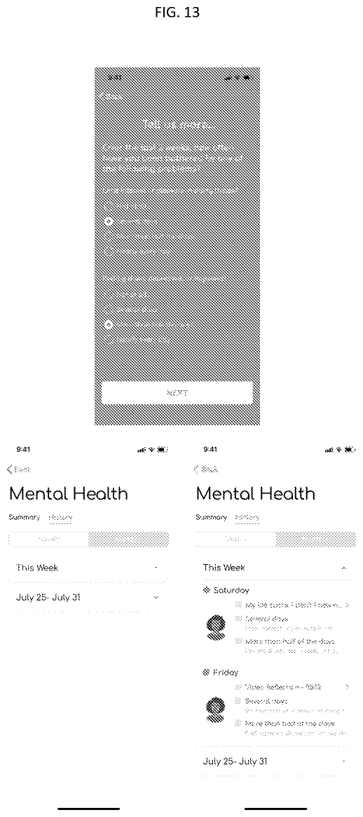

FIG. 13 shows an exemplary embodiment of a physical activity screen. In certain embodiments, the groups module comprises one or more of the following features selected from amongst Physical Activity Dashboard-Summary View, Physical Activity Dashboard-History View, Physical Activity Goal-Setting, Physical Activity Tracking. Support staff can determine how the algorithm decides what resources should be shown, and the criteria for determining which resources to show.

In certain embodiments, the Summary view will include the ability to initiate physical activity tracking. The Summary view will include the ability to see today's tracked physical activity. The Summary view will include the ability to see highlighted resources based on physical activity tracking history.

In certain embodiments, users will be able to understand how their physical activity is changing or staying constant over time. User will be able to view historical physical activity data (1 day, 1 week, 30 days/monthly).

In certain embodiments, user will have the default setting of tracking their physical activity for 1 week, but will be able to select any goal period (1,2.3 weeks or 1 month). The app will enable users to set a goal to track their physical activity for a period of time (1 week, 2 weeks, 3 weeks, or 1 month). Users will be able to set a min step goal of 3000 steps a day, and a max goal of 10000 steps a day. Users will be able to set a target daily step count goal. The support staff may provide guidance on acceptable ranges for goal setting in this category. Users will be able to set a target daily kilocal expenditure goal. The app will be recording physical activity tracking in the database and determining when the user has met their goal. Users will be able to set a target daily miles goal. Users will receive a badge when a physical activity monitoring or modification goal is met.

In certain embodiments, users will able to select specific support team members that they want to share their goal completion with. User will confirm share of information with support team member before sending. When a goal is met, users will have the option to share with their support team that they've met their goal. Users will have the option to share their progress towards their goal with their support team. Users will able to select specific support team members that they want to share their goals with. User will confirm share of information with support team member before sending. When setting a goal, users will be able to share that goal (e.g., “I want to track my physical activity for 2 weeks” or “I want to take 5000 steps a day”) with members of the support team. The app shall provide personalized support to users for goal setting in each health module. For example, in physical activity, incremental goals will be suggested for step counts based on the average weekly steps (e.g., a 10%, 20%, or 30% increase).

In certain embodiments, physical activity data will be pulled from Fibit Sense and Apple Health. The app will also be to enabled for automatic pulling of data from Android devices. Users will be able to automatically track their number of steps taken each day. In the event that automated Fitbit data pulling is not available or working, users will be able to manually enter their data as well. Users will be able to manually input steps taken each day. Requires Fitbit integration—this data will be pulled from Fibit Sense. Users will be able to track their miles traveled each day. Requires Fitbit integration—this data will be pulled from Fibit Sense. Users will be able to track their kilocal expenditure each day.

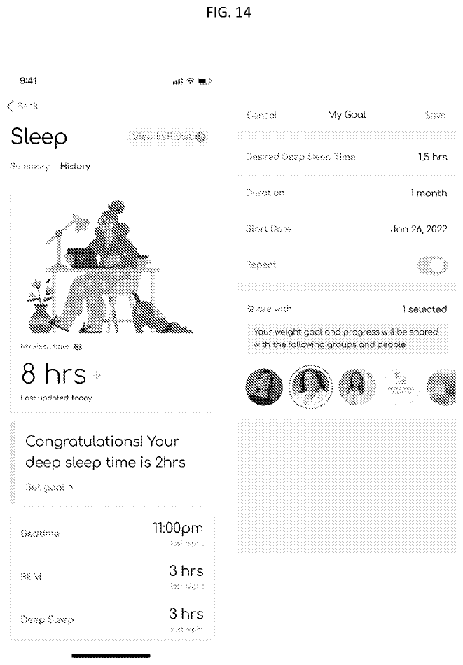

FIG. 14 shows an exemplary embodiment of a sleep screen. In certain embodiments, the groups module comprises one or more of the following features selected from amongst Sleep Dashboard—Summary View, Sleep Dashboard—History View, Sleep Goal—Setting, Sleep Tracking. Support staff can determine how the algorithm decides what resources should be shown, and the criteria for determining which resources to show.

In certain embodiments, the Summary view will include the ability to see today's tracked sleep. The Summary view will include the ability to see highlighted resources based on sleep tracking history.

In certain embodiments, users need to be able to understand how their sleep is changing or staying constant over time. User will be able to view historical sleep data. Users will be able to view a history of sleep journal entries.

In certain embodiments, user will have the default setting of tracking their sleep for 1 week, but will be able to select any goal period (1,2.3 weeks or 1 month). The app will enable users to set a goal to track their sleep for a period of time (1 week, 2 weeks, 3 weeks, or 1 month). Users will receive a badge when their sleep monitoring goal is met. Users will able to select specific support team members that they want to share their goal completion with. User will confirm share of information with support team member before sending. Users will have the option to share with their support team when they've met their goal. Users will have the option to share their progress towards their goal with their support team. Users will able to select specific support team members that they want to share their goals with. User will confirm share of information with support team member before sending. When setting a goal, users will be able to share that goal (e.g., “I want to track my sleep for 2 weeks”) with members of the support team.

In certain embodiments, the data will be pulled from Fibit Sense. Users will be able to automatically track and visualize their hours of sleep each day. Requires Fitbit integration—this data will be pulled from Fibit Sense. Data includes Bedtime, total #hours of sleep, #hours in REM, #hours in deep sleep. Users will be able to automatically track and visualize their sleep quality each day. In the event that automated Fitbit data pulling is not available or working, users should be able to manually enter their data as well. Users will be able to manually input number of hours slept each day. Journal entries can be made using text, audio, video. User will be able to create a journal entry reflecting on sleep.

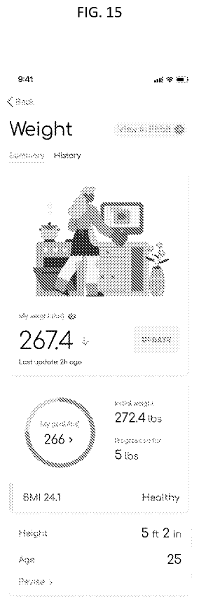

FIG. 15 shows an exemplary embodiment of a weight screen. In certain embodiments, the groups module comprises one or more of the following features selected from amongst Weight Dashboard-Summary View, Weight Dashboard-History View, Weight Goal-Setting, Weight Tracking. Support staff can determine how the algorithm decides what resources should be shown, and the criteria for determining which resources to show.

In certain embodiments, BMI will be calculated by CDC measures. The Summary view will include the ability to see today's tracked weight. The Summary view will include the ability to see highlighted resources based on weight tracking history.

In certain embodiments, user will be able to view historical weight data (1 day, 1 week, 30 days/monthly).

In certain embodiments, user will have the default setting of tracking their weight for 1 month, but will be able to select any goal period (1,2,3 weeks or 1 month). The app will enable users to set a goal to track their weight for a period of time (once a week for 1 week, 2 weeks, 3 weeks, or 1 month). The app will not permit weight goals that are below a healthy BMI range. The support staff may provide suitable thresholds. The app will enable users to set a numeric weight goal that is up to 5 pounds less than or greater than their current weight. The app will display a notification to users if they attempt to set a goal that is outside of a healthy range. Users will receive a badge when their weight monitoring or modification goals are met. Users will able to select specific support team members that they want to share their goal completion with. User will confirm share of information with support team member before sending. Users will have the option to share with their support team that they've met their goal. Users will have the option to share their progress towards their goal with their support team. Users will able to select specific support team members that they want to share their goals with. User will confirm share of information with support team member before sending. When setting a goal, users will be able to share that goal (e.g., “I want to track my weight for 2 weeks”) with members of the support team.

In certain embodiments, this data will be pulled from Fibit Sense. Users will be able to automatically track and visualize their weight each day. This data will be pulled from Fibit Sense. Users will be able to automatically track and visualize their BMI. Journal entries can be made using text, audio, video. User will be able to create a journal entry reflecting on their weight. In the event that automated Fitbit data pulling is not available or working, users should be able to manually enter their data as well. Users will be able to manually input their weight. The support staff will set parameters for the algorithm for determining if an alert message is needed, and the message to display (e.g., If user gains 5+ lbs. in 1 week post pregnancy the user will be prompted to monitor symptoms risk of preeclampsia). The app will display an alert message if users log data of clinical concern.

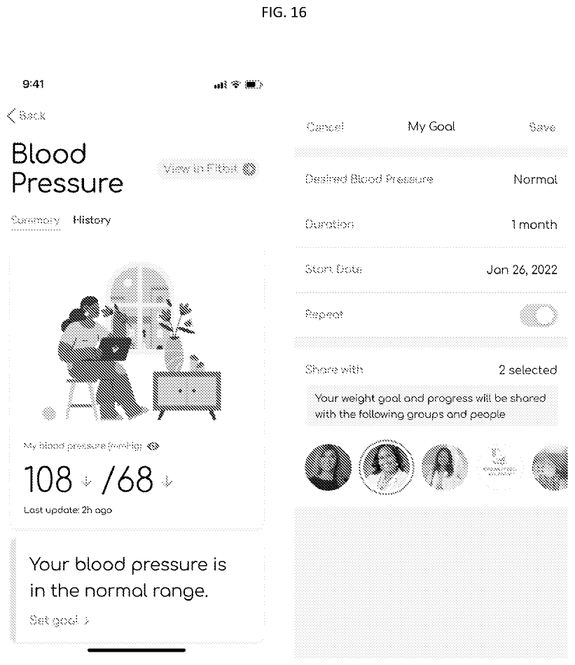

FIG. 16 shows an exemplary embodiment of a blood pressure screen. In certain embodiments, the blood pressure module comprises one or more of the following features selected from amongst Blood Pressure Dashboard-Summary View, Blood Pressure Dashboard-History View, Blood Pressure Goal-Setting, Blood Pressure Tracking. Support staff can determine how the algorithm decides what resources should be shown, and the criteria for determining which resources to show.

In certain embodiments, the Summary view will include the ability to see today's tracked blood pressure. The Summary view will include the ability to see highlighted resources based on blood pressure tracking history.

In certain embodiments, user will be able to view historical blood pressure data (1 day, 1 week, 30 days/monthly).

In certain embodiments, user will have the default setting of tracking their blood pressure for 1 month if the user indicated high blood pressure or preeclampsia in the health survey during account sign up. All other users the default will be 2 weeks. However, users will be able to select any goal period (1,2.3 weeks or 1 month). The app will enable users to set a goal to track their blood pressure for a period of time (daily for 1 week, 2 weeks, 3 weeks, or 1 month). Users will receive a badge when their blood pressure monitoring goal is met. Users will able to select specific support team members that they want to share their goal completion with. User will confirm share of information with support team member before sending. Users will have the option to share with their support team that they've met their goal. Users will have the option to share their progress towards their goal with their support team. Users will able to select specific support team members that they want to share their goals with. User will confirm share of information with support team member before sending. When setting a goal, users will be able to share that goal (e.g., “I want to track my blood pressure for 2 weeks”) with members of the support team.

In certain embodiments, in the event that automated data pulling is not available or working, users should be able to manually enter their data as well. Users will be able to manually input their blood pressure reading. Users will be able to automatically track blood pressure via a Bluetooth blood pressure cuff. The support staff may set parameters for the algorithm for determining if an alert message is needed, and the message to display (e.g., If user gains 5+ lbs. in 1 week post pregnancy the user will be prompted to monitor symptoms for preeclampsia risk). The app will display an alert message if users log data of clinical concern.

In certain embodiments, users will receive a prompt to celebrate or motivate a group member. For example, User 123 is making progress towards a weight maintenance goal. All other users who have set a weight goal will receive a notification that User 123 is making progress towards her goal. The other users are invited to send User 123 positive affirmations. They can choose from a pre-defined list of emojis, stickers, and/or pre-composed positive affirmations. Users will have the ability to send group members anonymous, celebratory, positive affirmations (emojis, stickers, and textual affirmations) when other users meet their goals.

All celebrations are sent anonymously. Users will not know the identity of the person they are sending a celebration to, and users will not know the identity of the users sending celebrations.

In certain embodiments, user will receive a prompt to encourage a group member. For example, User 123 is NOT making progress towards a weight maintenance goal. All other users who have set a weight goal (i.e., lose, gain, maintain weight) will receive a notification that User 123 is NOT making progress towards her goal. The other users are invited to send User 123 encouragement messages. They can choose from a pre-defined list of emojis, stickers, and/or pre-composed encouraging text messages. Users will have the ability to send group members encouragement (emojis, stickers, and textual affirmations) when other users are NOT meeting their goals.

All celebrations are sent anonymously. Users will not know the identity of the person they are sending a celebration to, and users will not know the identity of the users sending celebrations.

In certain embodiments, receiving celebration and encouragement messages. All celebrations and encouragements sent will be stored in a Positive Affirmations page, that enables users to see new affirmations and what they're for, as well as affirmations received in the past. Users will be able to view celebration and encouragement messages from users. Users will be notified when new celebrations or encouragements are received.

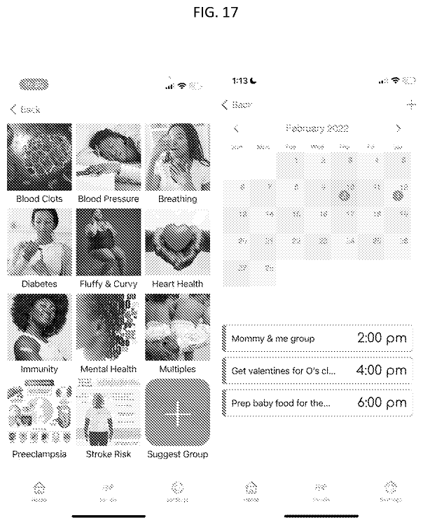

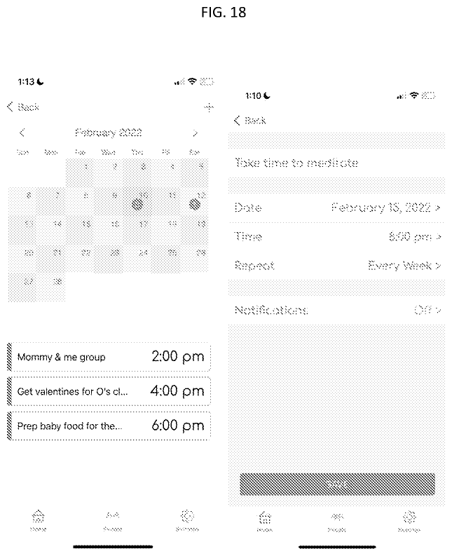

In certain embodiments, user will be able to create a new task on the panel page. The task can be a recurring daily/weekly/monthly. Users can create a new task to add to their task list. Users can mark tasks as “done”. In the daily task view, users can view completed tasks and those that are still open. User will be able to share tasks with Support Team. Users will be able to edit/update or delete a task. Users can view tasks in calendar view (seeing which days have tasks) (see FIG. 17). Users can view tasks in list view (seeing a list of tasks for each day) (see FIG. 18). Users receive an on-phone notification of upcoming or due task.

In certain embodiments, user will be able to add new support team member from phone contacts. The app would have the ability to access the user's phone contact. User can multi-select their contacts at once. User will be able to create new support team member by adding f name, l name, cell number, email address. User will be able to create new support team member. At least one pieces of contact information is needed. User will be able to share health data, goals, task/reminders with support team members. Goal are sharable from “health modules” and “task/reminders.” User will be able to add profile picture for support team members. User will be able to upload pictures. If they don't do that, then a default profile image is provided. User will be able to edit and delete a member by hard press their profile image listed. Support team members can be removed and edited.

In certain embodiments, additional profile question(s) have been developed, e.g., Users can answer the question “What do you love about being a black woman?” and have that information viewable on their profile (if they choose). The app allows the user to input information that will be shown on a publicly-viewable profile page.

In certain embodiments, in a user's profile setup and view mode, it should be clear what information is always viewable to others (username), always not viewable to others (birthdates), and any information they can choose to make viewable or not (bio, goals, . . . ). Users are able to indicate which information in their profile they want to be viewable to other users.

In certain embodiments, user sets up their account with first and last name. They should also be able to choose a “user name”, and that is the name that is shown to other users (not their first/last name). User is able to create a user name that will be viewable to other users.

In certain embodiments, user will be able to edit all profile info. Profile information is able to be edited

In certain embodiments, the algorithm will provide parameters for notification messages and notification triggers (e.g., after how many days of nonuse a message should be sent). Users will receive push notifications on their phones to encourage app use after periods of non-use. Users will be notified when new message are posted to their groups, and when new resources are available.

Users will receive push notifications on their phones to alert them of new app content available. Users will be able to set preferences for the frequency of notifications. If the user elects to have no push notifications, they will only see notifications when entering the app. Users can customize notification frequency in Settings menu.

The app uses machine learning to generate predictive insights for the user based on their self-monitoring data. For example, preemptive resource recommendation based on observed trends and trajectories, or suggestions for goal setting based on data analysis.

A healthcare provider may take action to either make an appointment with the user of the application or the user of the application may take action to make an appointment to see a healthcare provider. The end result of the collection of information, analysis of the information collected and generation of personalized insights is to motivate and implement human actions on the part of either the user of the application, the healthcare providers, or both.

### System for Implementing the Method

Another aspect of the present application relates to a system for implementing the method of the present application. In particular, after collection of information by the methods described herein, such information is placed in data storage prior to data analysis. One of ordinary skill will understand that the means or method of data storage is not limiting. One of ordinary skill will understand that a data storage architecture that creates databases for each of the areas of information described herein may be created. For example, a database for non-clinical background information, e.g., identity, race, sexual orientation, etc, may be created, as well as databases for clinical data, e.g., blood pressure records. The storage of non-clinical background information and its integration with stored clinical information by data analytics enables the application, systems, and methods described herein to effectively reach populations not otherwise reached by conventional approaches.

In some embodiments, the system comprises: (i) one or more computing devices in data communication with each other, each device having one or more computer processors, a data communication connection, and one or more tangible non-transitory computer-readable media accessible by the one or more computer processors, and (ii) a plurality of databases, including a personal information database, a health information database, and a resources database, wherein each database is stored in the one or more tangible non-transitory computer-readable media.

The system may be hosted on a server or series of servers, and these servers may be dedicated or cloud-based. The system may be replicated in embodiments. Embodiments may include various other systems similarly configured and may include and execute one or more subsystem components to perform functions described herein. The system may support one or more databases.

The user devices may create data connections with servers using wired, wireless, or network connections. Through these connections, the users can search for health information, trends in their personal data, message other users or healthcare providers, receive feedbacks from the computer system or from the healthcare provider, and execute any other functionality of the system. The user devices may be a desktop computer, a laptop computer, a tablet computer, a smartphone, a server, a personal digital assistant, a palmtop computer, or other portable computing device, or any combination of these devices capable of supporting a web browser or other type of application for interacting with the system. Alternatively, the user devices may be limited to a display device capable of displaying output without data processing and memory capabilities. Multiple user devices may be employed either separately or jointly, and a data communication between the devices can be implemented using wired, wireless, or network connections.

As another embodiment, the system may be implemented locally. Any user devices described, including a desktop computer, laptop computer, tablet, or mobile device, is suitable to support a local implementation. It is to be understood that the local device may have internet connectivity, and accordingly may have access to cloud-based and server-based system resources.

In one embodiment, the system includes a processor, for executing software instructions. Processor may be a single processor or multiple processors, configured as, for example, a bladed server or other known server configurations. Instructions for execution by the processor may be stored in the random access memory (RAM), accessible by the processor through bus, in secondary storage, in an external data source accessible via external data reader, or received from the Internet or other network via network connection. The processing by processor may be implemented in software, such as software modules, for execution by computers or other machines. Processor may execute one or more computer programs in order to provide the functions described in this specification. These computer programs preferably include instructions executable to perform the system functions and methods described above and illustrated in the figures herein. Such methods and the processing may be implemented in software, such as software modules, for execution by computers or other machines. The applications preferably provide graphical user interfaces (GUIs) through which users may view and interact with the system. The GUIs may be formatted, for example, as web pages in HyperText Markup Language (HTML), Extensible Markup Language (XML) or in any other suitable form for presentation on a display device depending upon applications used by users to interact with the system.

Secondary storage may include a hard disk drive, floppy disk drive, optical disk drive, or other types of non-volatile data storage, and the system may include an external data reader for external data, which may be any removable data medium, used if necessary. These components may be interconnected with processor via bus as well. The system may connect with a network, for example, the Internet, or other network, through network connection. This connectivity allows for communication with client devices or other computer systems to monitor and harvest data sources, receive user input, or communicate with other systems supporting the system. Network connection may accomplish this connection through the use of wired, wireless, or network connections.

In this embodiment, a user device contains sufficient input/output functionality to support the display of information to a user and that user's interaction with the model. Suitable input devices include a touch-screen, keyboard, mouse, cursor-control device, touch-screen, microphone, digital camera, video recorder or camcorder, and may be used to enter information into GUIs during the use of the system. Suitable output devices include any type of device for presenting visual information, such as, for example, a computer monitor or flat-screen display (or mobile device screen), and may include any other device for presenting output, including specialized devices to accommodate sensory-impaired users.

The system may store one or more database structures in secondary storage, for example, for storing and maintaining one or more databases, and other information necessary to perform the methods described herein. Alternatively, the system may include in-memory databases stored in RAM or a database in external data source accessible via data reader. Suitable databases include relational, non-relational, transactional, hierarchical, multi-dimensional (e.g., OLAP), object-oriented databases, and the like.

Examples of the system include one or more dedicated server computers, such as bladed servers, personal computers, laptop computers, notebook computers, palm top computers, network computers, mobile devices, or any processor-controlled device capable of executing a web browser or other type of application for interacting with the system. Although only one system is described in detail, system implementations may use multiple computer system or servers as necessary or desired to support the users and may also use back-up or redundant servers to prevent network downtime in the event of a failure of a particular server. In addition, although system is described with various components, one skilled in the art will appreciate that the server can contain additional or different components. In addition, although aspects of an implementation consistent with the above are described as being stored in memory, one skilled in the art will appreciate that these aspects can also be stored on or read from other types of computer program products or computer-readable media, such as secondary storage devices. The computer-readable media may include instructions for controlling system to perform a particular method, such as methods described above.

The computing environment of the present disclosure may include any number of computer or other processing systems (e.g., end-user systems, server systems, etc.) and databases or other repositories arranged in any desired fashion, where embodiments of the present disclosure may be applied to any desired type of computing environment (e.g., cloud computing, client-server, network computing, mainframe, stand-alone systems, etc.). The computer or other processing systems employed by the present disclosure may be implemented by any number of any personal or other type of computer or processing system (e.g., desktop, laptop, PDA, mobile devices, etc.), and may include any commercially available operating system and any combination of commercially available and custom software (e.g., browser software, communications software, server software, etc.). These systems may include any types of monitors and input devices (e.g., keyboard, mouse, head mounted displays, voice recognition, etc.) to enter and/or view information.

Cloud computing may provide internet-based computing, whereby shared servers provide resources, software, and data to computers and other devices on demand. For example, the cloud may be a cloud computing service that includes at least one server computing device, which may include a service abstraction layer and a hypertext transfer protocol wrapper over a server virtual machine instantiated thereon. The server computing device may be configured to parse HTTP requests and send HTTP responses.

Cloud computing uses the Internet and central remote servers to maintain data and applications. A cloud computing service may include a cloud server and cloud storage in communication with a portal e.g., web portal for receiving and transmitting content. Cloud computing can allow users to access and use applications without installation and access personal files at any computer with internet access. Cloud computing can allow for more efficient computing by centralizing storage, memory, processing and bandwidth. The cloud can provide scalable, on-demand computing power, storage, and bandwidth. The cloud storage can be a model of networked computer data storage where data is stored on multiple virtual servers, generally hosted by third parties.

It is to be understood that the software of the present disclosure may be implemented in any desired computer language and could be developed by one of ordinary skill in the computer arts based on the functional descriptions contained in the specification and flow charts illustrated in the drawings. Further, any references herein of software performing various functions generally refer to computer systems or processors performing those functions under software control. The computer systems of the present disclosure may alternatively be implemented by any type of hardware and/or other processing circuitry.

The various functions of the computer or other processing systems may be distributed in any manner among any number of software and/or hardware modules or units, processing or computer systems and/or circuitry, where the computer or processing systems may be disposed locally or remotely of each other and communicate via any suitable communications medium (e.g., LAN, WAN, Intranet, Internet, hardwire, modem connection, wireless, etc.). For example, the functions of the embodiments of the present disclosure may be distributed in any manner among the various end-user/client and server systems, and/or any other intermediary processing devices. The software and/or algorithms described above and illustrated in the flow charts may be modified in any manner that accomplishes the functions described herein. In addition, the functions in the flow charts or description may be performed in any order that accomplishes a desired operation.

The software of the present disclosure may be available on a non-transitory computer useable medium (e.g., magnetic or optical mediums, magneto-optic mediums, floppy diskettes, CD-ROM, DVD, memory devices, etc.) of a stationary or portable program product apparatus or device for use with stand-alone systems or systems connected by a network or other communications medium.

The communication network may be implemented by any number of any type of communications network (e.g., LAN, WAN, Internet, Intranet, VPN, etc.). The computer or other processing systems of the present disclosure may include any conventional or other communications devices to communicate over the network via any conventional or other protocols. The computer or other processing systems may utilize any type of connection (e.g., wired, wireless, etc.) for access to the network. Local communication media may be implemented by any suitable communication media (e.g., local area network (“LAN”), hardwire, wireless link, Intranet, etc.).

The system may employ any number of any conventional or other databases, data stores or storage structures (e.g., files, databases, data structures, data or other repositories, etc.) to store information. The database system may be implemented by any number of any conventional or other databases, data stores or storage structures (e.g., files, databases, data structures, data or other repositories, etc.) to store information. The database system may be included within or coupled to the server and/or client systems. The database systems and/or storage structures may be remote from or local to the computer or other processing systems, and may store any desired data.

In some embodiments, the system contains a machine learning module for maintaining and updating the abnormality point matrix.

The embodiments of the present disclosure may employ any number of any type of user interface (e.g., Graphical User Interface (“GUI”), command-line, prompt, etc.) for obtaining or providing information, where the interface may include any information arranged in any fashion. The interface may include any number of any types of input or actuation mechanisms (e.g., buttons, icons, fields, boxes, links, etc.) disposed at any locations to enter/display information and initiate desired actions via any suitable input devices (e.g., mouse, keyboard, etc.). The interface screens may include any suitable actuators (e.g., links, tabs, etc.) to navigate between the screens in any fashion.

The present disclosure concerns systems and/or computer program products. A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The computer program products may include a computer readable storage medium (or media) having computer readable program instructions thereon for causing a processor to carry out aspects of the present disclosure. More specifically, the processes and logic flows described in this disclosure can be performed by one or more programmable processors executing one or more computer programs to perform functions by operating on input data and generating output. The processes and logic flows can also be performed by, and apparatus can also be implemented as, special purpose logic circuitry, e.g., a field programmable gate array (“FPGA”) or an application specific integrated circuit (“ASIC”).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio player, a global positioning system (“GPS”) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

### Computer Readable Medium

The computer readable storage medium can be a tangible, non-transitory device that can retain and store instructions for use by an instruction execution device. The computer readable storage medium may be, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (“RAM”), a read-only memory (“ROM”), an erasable programmable read-only memory (EPROM or Flash memory), a static random access memory (“SRAM”), a portable compact disc read-only memory (“CD-ROM), a digital versatile disk (“DVD”), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network (“LAN”), a wide area network (“WAN”), a cloud computing system and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (“ISA”) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or WAN, or the connection may be made to an external computer (for example, through the Internet using an Internet Service Provider).

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified herein. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified herein.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified herein.

The examples below are included solely for illustrative purposes and are not limiting on the application described herein.

### EXAMPLES

### Example 1: Preliminary Clinical Trial Results

The study conducts an RCT of PM3 mobile app intervention compared to the standard of care for women. Women in the study are of reproductive age 18-45 years. Eligibility criteria for mothers includes: a) female, b) own a smart phone; c) aged 18-45; d) in third trimester or recently had a baby (<4 weeks postpartum); and e) can be contacted by phone or email after hospital discharge. The total sample is 340 women (170 for PM3, 170 for control group).

i. Study Procedures for PM3 Participants

PM3 participants are approached in their third trimester, given study information, then assessed for eligibility and willingness to participate. After the consent process, participants randomized to the PM3 group complete baseline assessments using a study device that employs an online survey platform, after which they are granted access to set up a PM3 account. The PM3 participants are trained on all features.

ii. Study Procedures for Control (Standard of Care) Participants

The analysis compares the control arm with maternal mortality prevention content and strategies delivered via PM3 mobile app versus a more traditional approach. Following randomization, participants assigned to the standard of care control receive an appropriate parallel to the PM3 intervention group. Following randomization, participants in the standard of care control (n=170) are provided with standard postpartum education in the locations where most women deliver. The control group does not have access to intervention content until study completion. Participants in the control group complete all assessments (baseline, 3, 6, 12 months post-enrollment to the study), blood pressure and weight, and provide hair samples at baseline and 3-month assessments. Participants are contacted to come to the study facility to complete data collection.

The completion of a visit and other clinical outcomes are confirmed by a medical chart review. The study sees an increase of postpartum visit attendance by 25%, for a 75% of the population attending a postpartum visit within 4-6 weeks postpartum. PM3 increases the effectiveness of postpartum discharge education and improves women's compliance with postpartum care recommendations using a mobile app.

### Example 2: Further Preliminary Trials

The study analyses improvements by users of PM3 in self-care agency, establishing an appointment with a primary care provider to manage comorbidities; self-monitoring comorbidities (i.e., take blood pressure) daily for 6 months; health-related quality of life (HRQOL); earlier depression screening and initiation of appropriate mental health treatment; and health care resource utilization, in comparison to standard of care protocols.

PM3 participants are trained on how to take their blood pressure. Furthermore, PM3 participants are asked to provide a hair sample for the determination of baseline cortisol levels. PM3 includes a Help section that provides tutorials on how to operate each feature and describe all relevant protocols.

Women are given a wearable technology and scale to assist with self-monitoring. Each participant has 6 months to engage with the app. During the study period, participants receive notifications to encourage the use of the app. PM3 includes a) integrated self-management and tailored health features; b) a support center based on social support gaps identified by women; c) a multimedia resource kit comprising information (e.g., videos, websites, podcasts) related to women's postpartum health and community resources; and maternal and reproductive health legislative policies that impact women. Baseline and 3, 6, and 12-month in-person re-assessments to measure study outcomes are conducted. Hair samples are collected for the biometric measurement of stress (cortisol) at baseline and 3 months. In month 6, participants will complete an exit interview on user experience. At month 12, all participants complete post-assessment surveys.

Postpartum depression is forecasted by hair cortisol levels, so the assessment of this biological indicator is extended to the postpartum transition period up to 3 months. In addition, a risk and resilience perspective is taken with the assessment of cortisol levels to understand the toll that stress can have on the mind and body in women who are disparately experiencing several chronic stressors.

Cortisol assessment procedure. All study participants in the control (standard of care) and intervention (PM3) arms provide hair samples at baseline and at 3 months postpartum. On average, hair grows approximately one cm per month, allowing for approximately three months of aggregated cortisol data to be analyzed from each sample as a 3 cm hair segment reflects cumulative cortisol secretion over the past 3 months.

Blood pressure (BP) self-monitoring. BP self-monitoring instructions are provided when patients are enrolled, and patients are expected to demonstrate understanding of how to take their own blood pressure and enter results into PM3. Using color-coded instructions, BP self-monitoring parameters are used to create algorithms within the app. For instance, when patients input their BP, they are prompted to retake the BP. If the second reading is outside the expected range, this automatically triggers a request for a third reading (taken after 5 minutes). Persistent high or low readings automatically trigger a message asking the participant to contact their obstetric provider or seek care at an emergency department. PM3 provides notifications to remind patients to take their BP. Participants are asked to take 1 BP measurement in the morning and 1 in the evening. Additionally, they are asked to enter 1 weight measurement weekly.

Use of PM3 increases self-care agency, establishing an appointment with a primary care provider to manage comorbidities; self-monitoring of comorbidities (i.e., take blood pressure) daily for 6 months; health-related quality of life (HRQOL); earlier depression screening and initiation of appropriate mental health treatment; and health care resource utilization, in comparison to standard of care protocols.

The terminology used herein was chosen to best explain the principles of the embodiments, the practical application or technical improvement over technologies found in the marketplace, or to enable others of ordinary skill in the art to understand the embodiments disclosed herein; it is to be understood that alternative terminology may be preferred and, therefore, it is the substance of the description, not the semantics, that should control the understanding of the invention.

One of ordinary skill will understand that the embodiments described herein above do not represent the full range of embodiments encompassed by the methods and systems described herein. The above description is for the purpose of teaching the person of ordinary skill in the art how to practice the object of the present application, and it is not intended to detail all those obvious modifications and variations of it which will become apparent to the skilled worker upon reading the description. It is intended, however, that all such obvious modifications and variations be included within the scope of the present application, which is defined by the following claims. The aspects and embodiments are intended to cover the components and steps in any sequence which is effective to meet the objectives there intended, unless the context specifically indicates the contrary.

What is claimed is:

1. A method for monitoring a subject for reducing maternal mortality, comprising the steps of
(a) receiving, via a user interface of an application executing on one or more computer processors, a self-surveillance module, wherein said self-surveillance module comprises inputs from the subject for personal information for a plurality of types of information selected from the types of information comprising doctor appointments, physiologic parameters, mental health survey results, indicators of social instability and personally recorded expression;
(b) storing, via the one or more computer processors, said personal information of said subject in a database accessible by said application, and accessible by said subject via said user interface of said application;
(c) receiving, via said user interface of said application executing on one or more computer processors, a data visualization dashboard, wherein said dashboard displays to said subject trends over time in said personal information stored in said database;
(d) receiving, via a user interface of an application executing on one or more computer processors, a goal-setting module, wherein said goal-setting module comprises inputs from the subject regarding personal performance targets that are desired to be achieved in connection with trends in those types of personal information stored in said database;
(e) assigning, via the one or more computer processors, an abnormality point value for any of a plurality of deviations from trends over time in said personal information stored in said database and an abnormality point matrix stored on a memory device accessible by the one or more computer processors, the abnormality point matrix being generated and continually updated by a machine learning module;
(f) determining, via the one or more computer processors, a total abnormality point value of said trends for said subject via the one or more computer processors, based on the sum total of abnormality point values in real-time, wherein when the total abnormality point value of said trends for said subject exceeds a pre-selected point value an abnormality alert is generated;
(g) notifying, via said user interface, said subject about the abnormality alert; and
(h) providing, via said user interface, health information messaging to said subject, wherein said health information messaging is tailored to undo said abnormality in said trends of said subject.

2. The method of claim 1, wherein said user interface is accessed by said subject on a mobile device.

3. The method of claim 1, wherein said user interface is accessed by said subject on a wearable device.

4. The method of claim 1, wherein said health information messaging to said subject comprises one or more type of messaging including self-tracking reminders, doctor appointment reminders, alerts to notify a healthcare provider and de-stress providers.

5. The method of claim 4, wherein said health information messaging is triggered automatically in response to trends in said personal information of said subject that indicates increased maternal mortality risk.

6. The method of claim 1, wherein said health information messaging is displayed on said user interface as a text alert to said subject.

7. The method of claim 1, wherein said health information messaging is displayed on said user interface as a representative symbol to said subject, wherein said symbol alerts said subject to availability of health information that is accessible via said user interface.

8. The method of claim 1, wherein the method further comprises the step of:
(i) receiving, via a user interface of an application executing on one or more computer processors, messages indicating that said subject is within a predefined distance of a healthcare provider and providing information regarding how said subject can travel to said healthcare provider.

9. The method of claim 8, wherein said messages include geo-location information for said healthcare provider.

10. The method of claim 1, wherein said self-surveillance module further comprises inputs from the subject for personal information for social support selected from the types of information comprising sleep deprivation, absence of assistance with baby or other children, and mood-related symptoms, wherein said subject inputs measurements of personal stress on a predefined measurement scale presented to said subject via said user interface.

11. The method of claim 10, said health information messaging comprises stress management strategies that respond to the trends in personal information for social support of said subject.

12. The method of claim 8, wherein the method further comprises the step of:
(j) receiving, via a user interface of an application executing on one or more computer processors, messages inviting said subject to join groups of other individuals using said application, wherein said groups are able to communicate amongst members of the group via the user interface.

13. The method of claim 1, further comprising the step of tracking over time, via the one or more computer processors and the total abnormality point values, the relative improvement or lack of improvement over time with respect to maternal mortality risk by said subject.

14. The method of claim 1, further comprising the step of providing a system comprising:
(i) one or more computing devices in data communication with each other, each device having one or more computer processors, a data communication connection, and one or more tangible non-transitory computer-readable media accessible by the one or more computer processors, and
(ii) a plurality of databases, including a personal information database and a health information messaging database, wherein a personal information database and a health information messaging database are each stored in the one or more tangible non-transitory computer-readable media.

15. The method of claim 14, wherein the plurality of databases further comprises a resources database.

16. The method of claim 14, wherein the resources database comprises multi-media information, wherein said multi-media information is accessible by said subject via said user interface.

17. A system for monitoring and reducing maternal mortality in a subject, comprising:
one or more computer processors; and
one or more tangible computer readable media accessible by the one or more computer processors, wherein the one or more tangible computer readable media comprise instructions that, when executed by the one or more processors, cause the one or more processors to perform:
(a) receiving, via a user interface of an application executing on one or more computer processors, a self-surveillance module, wherein said self-surveillance module comprises inputs from the subject for personal information for a plurality of types of information selected from the types of information comprising doctor appointments, physiologic parameters, mental health survey results, indicators of social instability and personally recorded expression;
(b) storing, via the one or more computer processors, said personal information of said subject in a database accessible by said application, and accessible by said subject via said user interface of said application;
(c) receiving, via said user interface of said application executing on one or more computer processors, a data visualization dashboard, wherein said dashboard displays to said subject trends over time in said personal information stored in said database;
(d) receiving, via a user interface of an application executing on one or more computer processors, a goal-setting module, wherein said goal-setting module comprises inputs from the subject regarding personal performance targets that are desired to be achieved in connection with trends in those types of personal information stored in said database;
(e) assigning, via the one or more computer processors, an abnormality point value for any of a plurality of deviations from trends over time in said personal information stored in said database and an abnormality point matrix stored on a memory device accessible by the one or more computer processors, the abnormality point matrix being generated and continually updated by a machine learning module;
(f) determining, via the one or more computer processors, a total abnormality point value of said trends for said subject via the one or more computer processors, based on the sum total of abnormality point values in real-time, wherein when the total abnormality point value of said trends for said subject exceeds a pre-selected point value an abnormality alert is generated;
(g) notifying, via said user interface, said subject about the abnormality alert; and
(h) providing, via said user interface, health information messaging to said subject, wherein said health information messaging is tailored to undo said abnormality in said trends of said subject.

18. The system of claim 17, wherein said user interface is accessed by said subject on a mobile device.

19. The system of claim 18, further comprising the step of tracking over time, via the one or more computer processors and the total abnormality point values, the relative improvement or lack of improvement over time with respect to maternal mortality risk by said subject.

20. The system of claim 17, wherein said health information messaging to said subject comprises one or more type of messaging including self-tracking reminders, doctor appointment reminders, alerts to notify a healthcare provider and de-stress providers.

21. The system of claim 20, wherein said health information messaging is triggered automatically in response to trends in said personal information of said subject that indicates increased maternal mortality risk.

22. The system of claim 17, wherein said health information messaging is displayed on said user interface as a text alert to said subject.

23. The system of claim 17, wherein said health information messaging is displayed on said user interface as a representative symbol to said subject, wherein said symbol alerts said subject to availability of health information that is accessible via said user interface.

24. The system of claim 17, wherein said self-surveillance module further comprises inputs from the subject for personal information for social support selected from the types of information comprising sleep deprivation, absence of assistance with baby or other children, and mood-related symptoms, wherein said subject inputs measurements of personal stress on a predefined measurement scale presented to said subject via said user interface.

25. The system of claim 17, said health information messaging comprises stress management strategies that respond to the trends in personal information for social support of said subject.

27. The system of claim 17, wherein the plurality of databases further comprises a resources database, wherein the resources database comprises multi-media information, wherein said multi-media information is accessible by said subject via said user interface.

28. A method for modifying behavior of a subject to reduce maternal mortality, comprising the step of using the system of claim 17.

29. A tangible non-transitory computer readable storage medium, comprising instructions that, when executed by a computer processor, cause the processor to:
(a) receiving, via a user interface of an application executing on one or more computer processors, a self-surveillance module, wherein said self-surveillance module comprises inputs from the subject for personal information for a plurality of types of information selected from the types of information comprising doctor appointments, physiologic parameters, mental health survey results, indicators of social instability and personally recorded expression;
(b) storing, via the one or more computer processors, said personal information of said subject in a database accessible by said application, and accessible by said subject via said user interface of said application;
(c) receiving, via said user interface of said application executing on one or more computer processors, a data visualization dashboard, wherein said dashboard displays to said subject trends over time in said personal information stored in said database;
(d) receiving, via a user interface of an application executing on one or more computer processors, a goal-setting module, wherein said goal-setting module comprises inputs from the subject regarding personal performance targets that are desired to be achieved in connection with trends in those types of personal information stored in said database;
(e) assigning, via the one or more computer processors, an abnormality point value for any of a plurality of deviations from trends over time in said personal information stored in said database and an abnormality point matrix stored on a memory device accessible by the one or more computer processors, the abnormality point matrix being generated and continually updated by a machine learning module;
(f) determining, via the one or more computer processors, a total abnormality point value of said trends for said subject via the one or more computer processors, based on the sum total of abnormality point values in real-time, wherein when the total abnormality point value of said trends for said subject exceeds a pre-selected point value an abnormality alert is generated;
(g) notifying, via said user interface, said subject about the abnormality alert; and
(h) providing, via said user interface, health information messaging to said subject, wherein said health information messaging is tailored to undo said abnormality in said trends of said subject.

30. The tangible non-transitory computer readable storage medium of claim 29, further comprising instructions that, when executed by a computer processor, cause the processor to:
(i) one or more computing devices in data communication with each other, each device having one or more computer processors, a data communication connection, and one or more tangible non-transitory computer-readable media accessible by the one or more computer processors, and
(ii) a plurality of databases, including a personal information database and a health information messaging database, wherein a personal information database and a health information messaging database are each stored in the one or more tangible non-transitory computer-readable media.
